(19) United States
(12) Patent Application Publication
(10) Pub. No.: US 2007/0232961 A1
(43) Pub. Date: Oct. 4, 2007

(54) Tooth movement tracking system

(76) Inventor: Huafeng Wen, Redwood City, CA (US)

Correspondence Address: PAUL HASTINGS JANOFSKY & WALKER LLP, 3579 VALLEY CENTRE DRIVE, SAN DIEGO, CA, 92130, US

(21) Appl. No.: 11/542,691

(22) Filed: Oct. 2, 2006

Related U.S. Application Data
(63) Continuation of application No. 11/013,147, filed on Dec. 14, 2004, now abandoned.

(51) Int. Cl. A61B 5/103

(52) U.S. Cl. 600/595

(57) ABSTRACT

Systems and methods are disclosed for determining movement of a tooth model from a first position to a second position by identifying one or more common features on the tooth model; detecting the position of the common features on the tooth model at the first position; detecting the position of the common features on the tooth model at the second position; and determining a difference between the position of each common feature at the first and second positions.

### BACKGROUND

During orthodontic treatments using removable dental appliances such as aligners, an orthodontist or dentist needs to determine the current tooth position to understand whether the treatment is on-track. Traditionally, the doctor relies on a physical model of the patient's teeth. The doctor/technician first takes the patient's dental impression. A dental plaster is then used to pour up a dental record. A variety of tools are used to cut each individual tooth plaster, and put them on a base of hot wax. Each tooth is then moved to its desired position. When the hot wax cools down, each tooth will be fixed in its position. During this process, the doctor has to eyeball whether he has made the right setup. Further, during treatment, if the teeth are not at their expected positions, new appliances may need to be fabricated to reflect the teeth's actual positions. Again, to ascertain the current positions of the teeth, the doctor has to eyeball the teeth's position.

As discussed in U.S. Pat. No. 6,820,025, a number of tracking systems are available to determine positions of objects. One type of tracking system known in the art is the so-called mechanical tracking system. Such systems use an artificial exo-skeleton, which is worn by the user of a synthetic environment (typically, a computer-created simulated environment). Sensors (e.g., goniometers) within the skeletal linkages of the exo-skeleton have a general correspondence to the actual joints of the user. Joint angle data is fed into kinematic algorithms that are used to determine body posture and limb position. However, since the exo-skeleton is worn by the user, other systems must be used to ascertain the position of the user within the simulated environment. Such systems are fraught with numerous drawbacks. For one, aligning the goniometers with the joints of a human body is difficult, especially with multiple degree of freedom (DOF) joints. Additionally, the joints of the exo-skeleton cannot perfectly replicate the range of motion of the joints of a human body. Thus, such technologies can provide only a rough approximation of actual body movement. Another limitation stems from the fact that human bodies are of different sizes and dimensions. As a result, the exo-skeleton must be recalibrated for each user. Yet another limitation is imposed by the encumbrance of the exo-skeleton itself. The weight and awkward configuration of the exo-skeleton prevent a human user from interacting with his environment in a natural manner. As a result, it is unlikely that the user will become immersed in the synthetic environment in the desired manner.

Another widely used system is a magnetic tracking system. In such systems a large magnetic field is generated and calibrated. The user has many small sensors mounted at various points on his body. The sensors are sensitive to the generated magnetic field. Thus, changes in position and orientation of the user's body with respect to the generated magnetic field can be detected by the magnetic sensors. Some of drawbacks of such systems include very short range and difficulty in calibrating the generated magnetic field. The short range stems from the fact that magnetic fields decrease in power inversely with the square of the distance from the generating source. This restricts the use of such systems to areas about the size of a small room. In order to use a larger working area, user movement must be modified or scaled in some manner. As a result, the magnitude and frequency of position and orientation errors increase rapidly. Additionally, the presence of ferromagnetic material (like the metal in belt buckles or weapons) distorts the generated magnetic fields. Additionally, the magnetic sensors pick up noise from other magnetic fields generated in or near the environment. Unfortunately, these distorting magnetic fields are commonplace, being easily generated by a plethora of devices, including computer monitors, fluorescent lighting, powered electrical wiring in the walls, as well as many other sources. Additionally, other sources of magnetic field error exist. Only with the aid of extremely detailed look-up tables can even moderately accurate measurements be obtained. Thus, magnetic tracking based on a generated magnetic field is subject to positional and orientation inaccuracies which are highly variable and unpredictable.

Another system for detecting position and orientation of a body uses so-called optical sensing. Optical sensing, in general, covers a large and varying collection of technologies. All of these technologies depend on the sensing of some type of light to provide position and orientation information. Consequently, all of these technologies are subject to inaccuracies whenever a required light path is blocked. Additionally, these technologies suffer from interference from other light sources. All of these optical sensing systems require specially prepared environments having the necessary emitters and sensors. This prevents widespread usage and presents a significant and expensive limitation.

Yet another approach is a tracking system using acoustic trackers. Like the previously described magnetic trackers, such systems are limited in range due to the inherent limitations of sound propagation. Additionally, the physics of sound limit accuracy, information update rate, and the overall range of an acoustic tracking system. Moreover, due to the relatively directional nature of sound, clear lines of sight must be maintained in order to obtain accurate readings.

### SUMMARY

Systems and methods are disclosed for determining movement of a tooth model from a first position to a second position by identifying one or more common features on the tooth model; detecting the position of the common features on the tooth model at the first position; detecting the position of the common features on the tooth model at the second position; and determining a difference between the position of each common feature at the first and second positions.

Advantages of the system include one or more of the following. The system automatically tracks the amount of movement of each individual tooth. This is done by putting the values of the movement in the computer. The motion tracking system determines the amount of movement per stage as well as the accuracy of movement. The system can also perform other operations required for dental appliance fabrication.

Other aspects and advantages of the invention will become apparent from the following detailed description and accompanying drawings which illustrate, by way of example, the principles of the invention.

### BRIEF DESCRIPTION OF THE DRAWINGS

The following detailed description of the embodiments of the invention will be more readily understood in conjunction with the accompanying drawings, in which:

FIG. 1 shows an exemplary process for determining and tracking tooth movements.

FIG. 2 shows an exemplary tooth having a plurality of markers or fiducials positioned thereon for automatic movement tracking.

### DESCRIPTION

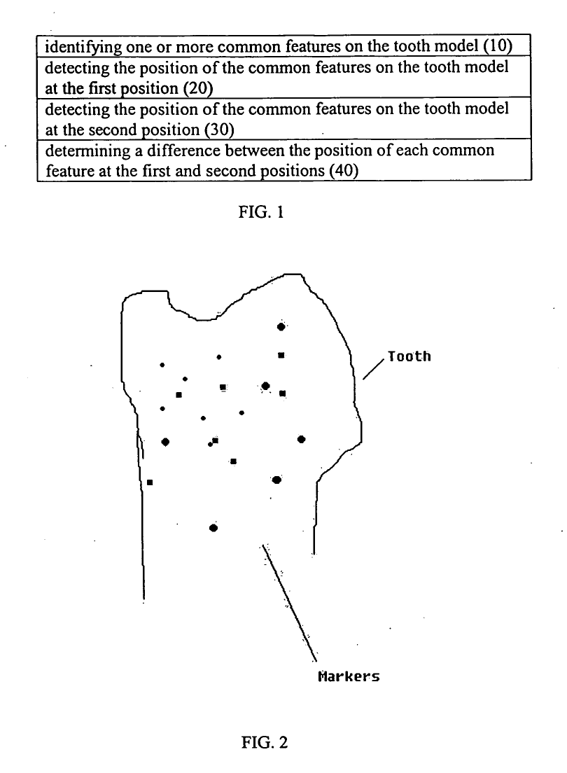

FIG. 1 shows an exemplary process for determining and tracking tooth movements. First, the process identifies one or more common features on the tooth model (10). Next, the process detects the position of the common features on the tooth model at the first position (20) and detects the position of the common features on the tooth model at the second position (30). The common features are constant and when measured on the tooth at the start represent the position of a tooth at the start (first) position. Correspondingly, when measured on the tooth at the current position, the common features represent the position of a tooth at the current (second) position. Having the start and current positions, the process determines a difference between the position of each common feature at the first and second positions (40)

In one embodiment, a mechanical based system is used to measure the position of the common features. First, the model of the jaw is placed in a container. A user takes a stylus and places the tip on different points on the tooth. The points touched by the stylus tip are selected in advance. The user then tells the computer to calculate value of the point. The value is then preserved in the system. The user takes another point until all points have been digitized. Typically, two points on each tooth are captured. However, depending on need, the number of points to be taken on each tooth can be increased. The points on all teeth are registered in computer software. Based on these points the system determines the differences between planned versus actual teeth position for aligner fabrication. These points are taken on each individual stage. In this way, this procedure can also be used to calculate the motion/movement of the tooth per stage.

Mechanical based systems for 3D digitization such as Microscribe from Immersion and Phantom from SenseAble Technology can be used. These 3D digitizers use counterbalanced mechanical arms (with a number of mechanical joints with digital optical sensors inside) that are equipped with precision bearings for smooth, effortless manipulation. The end segment is a pen like device called stylus which can be used to touch any point in 3D space. Accurate 3D position information on where the probe touches is calculated by reading each joint decoder information, 3D angular information can also be provided at an extra cost. In order to achieve true 6 degree of freedom information, an extra decoder can be added for reading pen self rotation information. Some additional sensors can be placed at the tip of the pen, so the computer can read how hard the user is pressing the pen. On the other side, a special mechanical device can be added to give force feedback to the user.

Immersion Corp.'s MicroScribe uses a pointed stylus attached to a CMM-type device to produce an accuracy of about 0.01 inch. It is a precision portable digitizing arm with a hand-held probe used at a workstation, mounted or on a tripod or similar fixture for field use or a manufacturing environment. The MicroScribe digitizer is based on optical angle encoders at each of the five arm joints, embedded processor, USB port and software application interface for the host computer. The user selects points of interest or sketches curves on the surface of an object with the hand-held probe tip and foot switch. Angle information from the MicroScribe arm is sent to the host computer through a USB or serial port. The MicroScribe utility software (MUS), a software application interface, calculates the Cartesian XYZ coordinates of the acquired points and the coordinates are directly inserted into keystroke functions in the user's active Windows application. The users design and modeling application functions are used to connect the 3D points as curves and objects to create surfaces and solids integrated into an overall design.

Another embodiment for 3D motion tracking/capture is based on optical or magnetic system. These require the model or the object that needs to be motion tracked to wear markers at specific points on the teeth and digitally recording the movements of the actual teeth so their movements can be played back with computer animation. The computer uses software to post-process this mass of data and determine the exact movement of the teeth, as inferred from the 3D position of each tooth marker at each moment.

In another embodiment, magnetic motion capture systems utilize sensors placed on the body to measure the low-frequency magnetic field generated by a transmitter source. The sensors and source are cabled to an electronic control unit that correlates their reported locations within the field. The electronic control units are networked with a host computer that uses a software driver to represent these positions and rotations in 3D space. Magnetic systems use 6 to 11 or more sensors per person to record body joint motion. The sensors report position and rotational information. Inverse kinematics (IK) is used to solve the angles for the various body joints, and compensate for the fact that the sensors are offset from the actual joint's center of rotation. The IK approach produces passable results from 6 sensor systems, but IK generally adds system overhead that can cause latency in real-time feedback. In this embodiment, sensors are applied to each individual tooth. Typically, three sensors are used: one on the buccal side, one on the lingual side and the one on the occlusal side. The number of sensors can be increased depending on the case.

In this embodiment, the jaw is placed in a housing or cabin. The sensors are attached to the teeth/jaw at predetermined points. These sensors are attached connected to an electronic system with the help of cables. The electronic system is in turn connected to a computer. The movement of the teeth at each stage is calculated by these sensors. The computer manipulates the coordinates and gives the proper values which are then used to perform the required procedures for aligner fabrication, among others.

Wireless sensors which operate at different frequencies can also be used. The movements are once again captured by electronics attached to the computer. With the help of the sensors, positional values are determined for aligner fabrication and other procedures that need to be performed.

In another embodiment, Optical Motion Capture Systems are used. There are two main technologies used in optical motion capture: Reflective and Pulsed-LED (light emitting diodes). Optical motion capture systems utilize proprietary video cameras to track the motion of reflective markers (or pulsed LEDs) attached to joints of the actor's body. Reflective optical motion capture systems use Infra-red (IR) LEDs mounted around the camera lens, along with IR pass filters placed over the camera lens. Optical motion capture systems based on Pulsed-LEDs measure the infra-red light emitted by the LED's rather than light reflected from markers. The centers of the marker images are matched from the various camera views using triangulation to compute their frame-to-frame positions in 3D space. A studio enclosure houses a plurality of video cameras (such as seven) attached to a computer. Dental impressions are placed inside the studio. Each of the teeth has a plurality of reflective markers attached. For example, markers can be placed on the buccal side, the lingual side and the occlusal side. More markers can be deployed if the tooth geometry is not constant or if required due to a particular situation in a case. Infra-red (IR) LEDs are mounted around the camera lens, along with IR pass filters placed over the lens. When the light emits form the LED's they gets reflected by the markers. The coordinates are captured and matched with the seven different camera views to ultimately get the position data for aligner making and other computations.

In an embodiment that uses chamfer matching, the system looks for a specific object in a binary image including objects of various shapes, positions, orientations. Matching is a central problem in image analysis and pattern recognition. Chamfer matching is an edge matching technique in which the edge points of one image are transformed by a set of parametric transformation equations to edge points of a similar image that is slightly different. In this embodiment, digital pictures of the jaw are taken from different angles (such as seven angles for each stage). Those pictures are taken at a plurality of different resolutions such as four resolutions. In one embodiment, a hierarchical method for computing the analysis compares all the pictures of one stage with all the pictures of the other stage. The chamfer matching operation then determines the total amount of movement of the teeth per stage. The movement of individual tooth can then be used for calculating information required for aligner fabrication.

In an embodiment that uses ‘laser marking’, a minute amount of material on the surface of the tooth model is removed and colored. This removal is not visible after the object has been enameled. In this process a spot shaped indentation is produced on the surface of the material. Another method of laser marking is called ‘Center Marking’. In this process a spot shaped indentation is produced on the surface of the object. Center marking can be ‘circular center marking’ or ‘dot point marking’.

In the laser marking embodiment, small features are marked on the crown surface of the tooth model. After that, the teeth are moved, and each individual tooth is superimposed on top of each other to determine the tooth movement. The wax setup is done and then the system marks one or more points using a laser. Pictures of the jaw are taken from different angles. After that, the next stage is produced and the same procedure is repeated. Stages x and x+1 pictures are overlaid. The change of the laser points reflects the exact amount of tooth movement.

In yet another embodiment called sparkling, marking or reflective markers are placed on the body or object to be motion tracked. The sparkles or reflective objects can be placed on the body/object to be motion tracked in a strategic or organized manner so that reference points can be created from the original model to the models of the later stages. In this embodiment, the wax setup is done and the teeth models are marked with sparkles. Alternatively, the system marks or paints the surface of the crown model with sparkles. Pictures of the jaw are taken from different angles. Computer software determines and saves those pictures. After that, the teeth models are moved. Each individual tooth is mounted on top of the other and tooth movement can be determined. Then the next stage is performed, and the same procedure is repeated.

In another embodiment that uses freehand without mechanical attachment or any restrictions, the wax setup operation is done in freehand without the help of any mechanical or electronic systems. Tooth movement is determined manually with scales and/or rules and these measurements are entered into the system.

An alternative is to use a wax set up in which the tooth abutments are placed in a base which has wax in it. One method is to use robots and clamps to set the teeth at each stage. Another method uses a clamping base plate. i.e. a plate on which teeth can be attached on specific positions. Teeth are setup at each stage using this process. Measurement tools such as the micro scribe are used to get the tooth movements which can be used later by the universal joint device to specify the position of the teeth.

In another embodiment, the FACC lines are marked. Movement is determined by non mechanical method or by a laser pointer. The distance and angle of the FACC line reflects the difference between the initial position and the next position on which the FAC line lies.

In a real time embodiment, the teeth movements are checked in real time. The cut teeth are placed in a container attached to motion sensors. These sensors track the motion of the teeth models in real time. The motion can be done with freehand or with a suitably controlled robot. Stage x and stage x+1 pictures are overlaid, and the change of the points reflects the exact amount of movement.

The system has been particularly shown and described with respect to certain preferred embodiments and specific features thereof. However, it should be noted that the above described embodiments are intended to describe the principles of the invention, not limit its scope. Therefore, as is readily apparent to those of ordinary skill in the art, various changes and modifications in form and detail may be made without departing from the spirit and scope of the invention as set forth in the appended claims. Other embodiments and variations to the depicted embodiments will be apparent to those skilled in the art and may be made without departing from the spirit and scope of the invention as defined in the following claims.

In particular, it is contemplated by the inventor that the principles of the present invention can be practiced to track the orientation of teeth as well as other articulated rigid bodies including, but not limited to prosthetic devices, robot arms, moving automated systems, and living bodies. Further, reference in the claims to an element in the singular is not intended to mean “one and only one” unless explicitly stated, but rather, “one or more”. Furthermore, the embodiments illustratively disclosed herein can be practiced without any element which is not specifically disclosed herein.

1. A method for determining movement of a tooth model from a first position to a: second position, comprising:
identifying one or more common features on the tooth model;
detecting the position of the common features on the tooth model at the first position;
detecting the position of the common features on the tooth model at the second position; and
determining a difference between the position of each common feature at the first and second positions.

2. The method of claim 1, comprising:
forming the model of the tooth at the first position; and
forming the model of the tooth at the second position.

3. The method of claim 1, wherein the position detecting comprises mechanically sensing the position.

4. The method of claim 3, comprising using handheld 3D digitizers.

5. The method of claim 1, wherein the position detecting comprises chamfer matching.

6. The method of claim 1, wherein the position detecting comprises sparkling.

7. The method of claim 1, wherein the position detecting comprises laser marking a tooth model.

8. The method of claim 1, wherein the position detecting comprises marking an FACC line.

9. The method of claim 1, wherein the position detecting comprises manually measuring the difference between first and second positions.

10. The method of claim 1, wherein the position detecting comprises setting up a wax model.

11. A system for determining movement of a tooth model from a first position to a second position, comprising:
means for identifying one or more common features on the tooth model;
means for detecting the position of the common features on the tooth model at the first position;
means for detecting the position of the common features on the tooth model at the second position; and
means for determining a difference between the position of each common feature at the first and second positions.

12. The system of claim 11, comprising:
means for forming the model of the tooth at the first position; and
means for forming the model of the tooth at the second position.

13. The system of claim 11, wherein the position detecting comprises mechanically sensing the position.

14. The system of claim 13, comprising using handheld 3D digitizers.

15. The system of claim 11, wherein the position detecting means comprises chamfer matching means.

16. The system of claim 11, wherein the position detecting means comprises sparkling means.

17. The system of claim 11, wherein the position detecting means comprises means for laser marking a tooth model.

18. The system of claim 11, wherein the position detecting means comprises means for marking an FACC line.

19. The system of claim 11, wherein the position detecting means comprises means for manually measuring the difference between first and second positions.

20. The system of claim 11, wherein the position detecting means comprises a jig for wax model set-up.
